(19) United States
(12) Patent Application Publication
(10) Pub. No.: US 2009/0149730 A1
(43) Pub. Date: Jun. 11, 2009

(54) Temperature probe unit

(75) Inventor: Craig R McCrary, Valencia, CA (US)

Correspondence Address: WILLIAM W. HAEFLIGER, 201 S. LAKE AVE, SUITE 512, PASADENA, CA, 91101, US

(73) Assignee: Neotech Products, Inc.

(21) Appl. No.: 11/999,405

(22) Filed: Dec. 6, 2007

(51) Int. Cl. A61B 5/01

(52) U.S. Cl. 600/392; 600/391

(57) ABSTRACT

A sensor means in the form of a substantially flat package for receiving and transmitting temperature indicating signals, to be monitored, comprising in combination, a first layer in the form of an annulus having an undersurface to adhere to the skin of a patient, a second layer in the form of a conductive sensing layer peripherally bounded by said first layer to block leakage from the second layer, and having an undersurface exposed downwardly for sealing contact with the skin of the patient, a foam layer extending above the first and second layers in adjacent relation therewith, a reflective layer extending over the foam layer, and an electrically conductive lead extending in contact with at least one of the first and second layers, and to the package exterior, as for controlling a warmer.

### BACKGROUND OF THE INVENTION

This invention relates generally to sensors attachable to the skin of a patent, such as an infant, and more particularly to improvements in sensor packages for topically receiving and transmitting physiologic signals such as skin temperature, for controlling warming device.

There is need for improvements in such devices, particularly as related to achieving flat package configurations, skin protection prevention of leakage, improved adhesion to fragile skin, protection of electrical components, capability to efficiently respond to body temperature, via the skin, in a non-invasive manner, for warming control, monitoring or recording, and ease and reliability of use, as well as other objectives and advantages in construction as will be seen.

### SUMMARY OF THE INVENTION

It is a major object of the invention to provide improvements in such sensor packages and their use meeting the above needs.

Basically, the sensor package of the invention comprises, in combination:

a) a first layer in the form of an annulus having an undersurface to adhere to the skin of a patient,

b) a second layer in the form of a conductive sensing layer peripherally bounded by the first annular layer to block leakage from the second layer, and having an undersurface exposed downwardly for sealing contact with the skin of the patient,

c) a foam layer extending above the first and second layers in adjacent relation therewith,

d) a reflective layer extending over the foam layer,

e) and an electrically conductive lead extending in contact with at least one of the first and second layers, and to the package exterior.

Additional objects include locating the second layer undersurface in the same plane as defined by a lower annular sealing surface of the first layer; provision of a strip-off protective layer removably attached to undersurfaces of the first and second layers, initially preventing such leakage; and provision of a warmer controlling connected to the conductive lead, outside the package. Also, adhesive material is typically provided between the foam layer and the first and second layer, whereby the foam layer orients the first and second layers in side by side relation with their undersurfaces in a common plane. Adhesive material may also extend between the foam and reflective layers whereby the heat reflective layer contributes to the functioning of the foam layer, as well as blocking heating of the foam and first and second layers from external heat sources.

These and other objects and advantages of the invention, as well as the details of an illustrative embodiment, will be more fully understood from the following specification and drawings, in which:

### DRAWING DESCRIPTION

FIG. 1 shows the invention and its components, in preferred form; and

FIG. 2 is a plan view of an electrical lead and temperature sensor.

### DETAILED DESCRIPTION

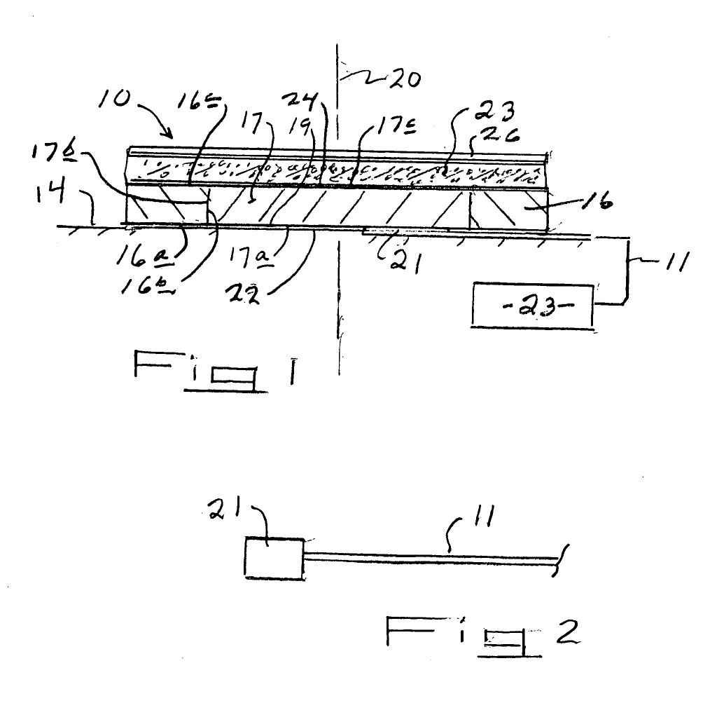

FIG. 1 shows sensor means, in the form of a package or assembly 10 for receiving and transmitting signals such as temperature control signals, to be monitored. A lead 11 extends from the package to a warmer 23.

The package sensor assembly 10 includes a first layer 16 as for example in the form of an annulus extending about a central axis 20, and having an undersurface 16a to adhere to the patient's skin 14 under manual pressure transmission downwardly on the package. The annulus 16 preferably comprises a hydrocolloid material, such materials being known.
Examples are:
DuoDerm CGF Control Formula Dressing
Cutinova Hydro
3 M Tegasorb Hydrocolloid Dressing
Replicare

Package 10 also includes a second layer 17 in the form of sensing gel material, peripherally bounded by layer 16, the bottom surfaces 17a and 16a being in the same plane. The gel may be electrically conductive. Undersurface 17a is adapted for contact with the skin of the patient or infant, along with undersurface 16a, since both 16 and 17 are flexible, considering that the skin may not necessarily be locally flat (but may be typically upwardly convex) at the locus of application of the package to the skin. The gel material of 17 is known as Hydrogel and known examples of such material are identified as follows:
Hypergel
Intra Site Gel
Normigel
MKM Gentell Hydrogel

Electrical lead 11 extends generally radially, directly and locally under the lower surfaces 16a and 17a, as shown, from a temperature sensor 21, in the form of a transducer whose electrical output is a function of skin or body surface 14 temperature. Lead 11 extends to warming device 23 proximate the body of the patient or infant, the heating output of 23 being controlled by the electrical output of 21, so as to produce heating if the sensed skin or body temperature falls below a predetermined level.

An important function of the annular layer 16 is to block leakage of moisture from the layer 17 to the exterior, as via adjacent annular sides 16b and 17b. In that regard, a thin layer of adhesive is preferably employed or adhered at 19 to the layer undersides 16a and 17a, that layer 19 underlying those undersides to peripherally block leakage from 17 at underside 16a of layer 16. One such adhesive is PSA (Pressure Sensiting Adhesive). A strip-off protective layer 22 of material, such as a plastic film initially extends under the entire adhesive layer 19, and also beneath undersurfaces 16a and 17a, to protect the assembly until it is applied to the skin.

Also shown in FIG. 1 is an insulative layer 23, such as a foam plastic material, which extends over the entire top surfaces 16c and 17c of the layers 16 and 17. The undersurface of layer 23 is adhesively bonded at 24 to 16c and 17c, and layer 23 functions to block heat transfer upwardly from 16 and 17. Also, layer 23 assists in stabilizing the relative positions of 16 and 17. A thin reflective layer 26 such as a film, extends over and attaches to the foam layer, to reflect impinging radiation, such as heat, so that such radiation will not affect the body or skin temperature sensing function of the device. Typically, the film has a reflective, very thin, gold surface. The plastic material of layer 23 may typically consist of PVC Foam.

The dimensions of the first layer 16 are approximately as follows:

| | | |
|---|---|---|
| thickness | .012 | inches |
| inner diameter | .69 | inches |
| outer diameter | 1.0 | inch |

The dimensions of the second layer 17 are approximately as follows:

| | |
|---|---|
| thickness | .12 inch |
| overall diameter | .69 inch |

The dimensions of the foam layer 23 are approximately as follows:

| | |
|---|---|
| thickness | .094 inch |
| overall diameter | 1.0 inch |

The thickness of the reflective layer 26 (such as gold Mylar foil) is approximately as follows:

thickness 0.02 inch.

It is found that these dimensions materially assist in optimizing the operation of the device of FIG. 1, considering application to the skin of infants.

1. A sensor means in the form of a substantially flat package for receiving and transmitting signals, to be monitored and/or recorded, comprising in combination:
a) a first layer in the form of an annulus having an undersurface to adhere to the skin of a patient,
b) a second layer in the form of a conductive sensing layer peripherally bounded by said first layer to block leakage from the second layer, and having an undersurface exposed downwardly for sealing contact with the skin of the patient,
c) a foam plastic layer extending over the entire top surfaces of the first and second layers and adhesively bonded to said top surfaces whereby the foam plastic layer blocks heat transfer upwardly from the first and second layers, in adjacent relation therewith,
d) a reflective layer extending over the foam layer, to reflect impinging heat radiation away from the foam layer,
e) an electrically conductive lead extending under said first and second layers and in contact with at least one of the first and second layers, and also extending to the package exterior,
f) said foam plastic layer assisting in stabilizing the relative positions of the first and second layers, above the level of the lead.
g) a thin layer of adhesive adhered to the undersides of said first and second layers,
h) and a strip-off protective plastic film layer extending under the entire adhesive layer, beneath said first and second layers undersides.

2. The combination of claim 1 wherein said first layer consists of a hydrocolloid material.

3. The combination of claim 1 wherein said second layer undersurface is in the same plane as defined by a lower annular surface of the first layer.

4. (canceled)

5. The combination of claim 1 wherein said packages has layered configuration, and including a warmer connected to said lead, outside the package.

6. The combination of claim 1 wherein said reflective layer consists of gold.

7. The combination of claim 1 including adhesive material between said foam layer and said first and second layers, and adhesive material between said foam layer and said reflective layer.

8. The combination of claim 1 wherein the dimensions of said first layer are approximately as follows:

| | | |
|---|---|---|
| thickness | .012 | inches |
| inner diameter | .069 | inches |
| outer diameter | 1.0 | inch. |

9. The combination of claim 8 wherein the dimensions of said second layer are approximately as follows:

| | |
|---|---|
| thickness | .12 inch |
| overall diameter | .69 inch. |

10. The combination of claim 9 wherein the dimensions of said foam layer are approximately as follows:

| | |
|---|---|
| thickness | .094 inch |
| overall diameter | 1.0 inch. |

11. The combination of claim 9 wherein the dimensions of said reflective layer are approximately as follows:

| | |
|---|---|
| thickness | .02 inch. |
